(19) United States
(12) Patent Application Publication
(10) Pub. No.: US 2021/0077834 A1
(43) Pub. Date: Mar. 18, 2021

(54) MULTI-FREQUENCY ULTRASOUND TRANSDUCERS

(71) Applicants: Kobi VORTMAN, Haifa (IL); Shuki VITEK, Haifa (IL)

(72) Inventors: Kobi VORTMAN, Haifa (IL); Shuki VITEK, Haifa (IL)

(21) Appl. No.: 16/959,914

(22) PCT Filed: Jan. 4, 2019
(86) PCT No.: PCT/IB2019/000033

§ 371 Date: Jul. 2, 2020

Related U.S. Application Data
(60) Provisional application No. 62/613,890, filed on Jan. 5, 2018.

(51) Int. Cl. A61N 7/00; A61B 5/055; A61B 5/107; A61B 5/00

(52) U.S. Cl. CPC A61N 7/00; A61B 5/055; A61B 5/1075; A61N 2007/0091; A61B 5/4836; A61N 2007/0073; A61B 5/4887

(57) ABSTRACT

Treatment of target tissue in a target volume having multiple target regions includes causing an ultrasound transducer to transmit a first series of ultrasound waves having a first frequency to a first one of target regions; and causing the ultrasound transducer to transmit a second series of ultrasound waves having a second frequency, different from the first frequency, to a second one of the target regions, different from the first one of the target regions, based on one or more different anatomical characteristics (such as focal lengths) between the first and second ones of the target regions.

### RELATED APPLICATION

This application claims the benefit of and priority to U.S. Provisional Patent Application No. 62/613,890, filed Jan. 5, 2018, the entire disclosure of which is hereby incorporated by reference.

### FIELD OF THE INVENTION

The present invention relates, generally, to ultrasound systems. In particular, various embodiments are directed to ultrasound transducers capable of transmitting waves at multiple frequencies.

### BACKGROUND

Focused ultrasound (i.e., acoustic waves that have a frequency greater than about 20 kHz and may be focused to a point in space) can be used to image or therapeutically treat internal body tissues within a patient. For example, ultrasonic waves may be used to ablate tumors, eliminating the need for the patient to undergo invasive surgery. For this purpose, a piezo-ceramic transducer may be placed externally to the patient, but in close proximity to the tissue to be ablated (“the target”). The transducer converts an electronic drive signals into mechanical vibrations, resulting in the emission of acoustic waves. The transducer may be shaped so that the emitted waves converge in a focal zone. Typically, the transducer functions in a vibrational mode along the acoustic emission direction. In some cases, the acoustic emission may include shear waves propagating in the shear mode. Single-plate transducers tend to have power-delivery efficiencies of 50%-60% and a bandwidth of approximately ±10% of the center frequency. Single-transducer designs have advantages such as low cost and efficient power transmission. But in cases where the transducer element has linear dimensions larger than the wavelength of the transmitted waves, the focal-zone steering angles will be very limited.

Alternatively, the transducer may be formed of a two-dimensional grid of uniformly shaped piezoelectric transducer elements that can be glued, via a polymer matrix, to a matching conductive substrate. For example, each of the elements may be a single “rod” or multiple “rods” that have been joined together. Typically, each transducer element transmits acoustic waves along the direction of rod elongation and can be driven individually or in groups; thus the phases of the transducer elements can be controlled independently. Such a “phased-array” transducer facilitates focusing the transmitted energy into a focal zone and steering the focus to different locations by adjusting the relative phases among the transducer elements and/or simultaneously generating multiple foci to treat multiple target sites by grouping the transducer elements. Phased-array transducers may have bandwidths of 30%-40% of the center frequency, but are less capable of high-power transmission (compared with the single-plate transducer) due to the poor thermal stability and low thermal conductivity of the polymer matrix. In addition, because the intensity at the third harmonic of the transducer resonant frequencies may be damped by the polymer matrix, the high-bandwidth phased-array transducer typically cannot transmit sufficient power at a frequency above the base harmonic.

It has been shown that transducers having a multilayer structure with no functional layers outside the two electrode layers of the transducer (i.e., “air-backing transducers”) may provide a high power-delivery efficiency. These transducers, however, suffer from narrow frequency bandwidths (e.g., less than ±5% or ±10% of the center frequency). A wide bandwidth is particularly preferred in ultrasound treatment applications because it offers a large range of frequencies that may be optimized for different depths in tissue, facilitating treatment at different target regions. Accordingly, there is a need for an approach that provides a high-power ultrasound output for treatment while retaining the ability to treat different target regions.

### SUMMARY

Embodiments of the present invention provide an ultrasound system that can deliver a high-power output with two or more frequencies (such as 1.2 MHz and 3 MHz) to a target volume. In various embodiments, the target volume is divided into multiple regions; the transducer directs ultrasound waves having different frequencies to different regions of the target volume. For example, waves having a high frequency (e.g., 3 MHz) may be directed to proximal target region(s) corresponding to short focal length(s), while waves having a low frequency (e.g., 1.2 MHz) may be directed to distal target region(s) corresponding to long focal length(s). Because the frequency of ultrasound waves applied to each target region is optimized for obtaining the maximum power absorption in the focal zone therein, utilizing different frequencies to treat different target regions may advantageously optimize the overall ultrasound treatment effect at the target. Typically, as the focal depth increases, the absorption of the acoustic power in the path zone (i.e., the zone through which the acoustic beam propagates to the target) increases; as a result, the power arriving at the focal zone after propagating through the path zone decreases, so the power absorption in the focal zone also decreases. The reduced power absorption at the focal zone can be compensated for by adjusting the frequency of the applied waves, taking into account the focal depth in the tissue and the power absorption in the path zone and focal zone. In some embodiments, the ultrasound frequency may be varied based on real-time feedback of the temperature and/or other characteristic(s) measured at the target and/or non-target regions. For example, a high frequency may be utilized first to initiate treatment; upon detection of overheating at a non-target region in the near field, the system may switch to a low-frequency mode for treatment so as to avoid damage to the non-target tissue. Accordingly, adjustments of the ultrasound frequency may allow the acoustic power to be efficiently absorbed in dynamically selected regions of the target volume, thereby optimizing treatment and avoiding undesired damage to the non-target tissue.

Variation of the ultrasound frequency may also change the size of the focal zone, thereby affecting the peak acoustic intensity therein. Generally, at a given focal depth, increasing the ultrasound frequency decreases the size of the focal zone, which in turn increases the peak intensity at the focal zone. Therefore, at a certain focal length, the ultrasound frequency of the applied waves may reflect a trade-off between the absorption of acoustic power in the path zone, the power absorption at the target and the peak intensity at the focal zone. Accordingly, in some embodiments, the ultrasound frequency associated with each target region in the target volume is optimized based on anatomical characteristics (e.g., the tissue type, size, location, tissue structure, thickness, density, vascularization, etc.) of the tissue therein so as to achieve a desired treatment effect. For example, highly vascular tissue may have a low absorption coefficient; in this case, the tissue will tolerate high energy levels, enabling the use of high ultrasound frequencies in order to increase absorption at the distal target region without adverse effect on tissue surrounding the proximal target region.

Further, the steering capability of the ultrasound beam may be tuned via adjustment of the ultrasound frequency. As described in greater detail below, the ultrasound beam is steered through phase adjustments of the transducer element emissions, exploiting constructive and destructive interference among the waves propagating from the different elements. Typically, a higher frequency corresponds to a more accurate but more limited (in terms of maximum angular deflection) steering capability. Therefore, in one embodiment, the high-frequency waves are employed for treatment when highly accurate steering is desired and the corresponding limited steering capability (e.g., steering angle <)±10° is acceptable. The low-frequency waves may be utilized when a large steering angle (e.g., steering angle >)±30° is preferred or needed. Accordingly, via adjustment of the ultrasound frequency, transducers in accordance herewith may provide steering capabilities tailored to a particular ultrasound procedure. This approach may advantageously obviate the need for the mechanical steering mechanisms or combination of electronic and mechanical steering implemented in conventional ultrasound therapy systems.

Accordingly, in one aspect, the invention pertains to a system for treating target tissue in a target volume having multiple target regions, In various embodiments, the system includes an ultrasound transducer for transmitting ultrasound waves having two or more frequencies; and a controller configured to cause the ultrasound transducer to transmit the first series of ultrasound waves having the first frequency to the first one of target regions; and cause the ultrasound transducer to transmit the second series of ultrasound waves having the second frequency, different from the first frequency, to the second one of the target regions, different from the first one of the target regions, based on one or more different anatomical characteristics between the first and second ones of the target regions. In one embodiment, the first frequency is higher than the second frequency and the anatomical characteristic is relative location; the location of the first target region corresponds to a shorter focal depth of the transducer than that of the second target region. In another embodiment, the first frequency is higher than the second frequency and the anatomical characteristic is vascularization; the first target region has higher vascularity than the second target region.

In various embodiments, the system further includes a monitoring system (e.g., an MRI apparatus) for measuring the anatomical characteristic(s) (e.g., the type, size, location, property, structure, thickness, density, and/or vascularization of tissue) associated with one or more target regions and/or a non-target region. In addition, the system may further include memory for storing a treatment plan specifying the anatomical characteristic(s) and parameter values (e.g., the frequency, phase, amplitude and/or sonication duration) associated with the ultrasound transducer for transmitting the first series and second series of ultrasound waves based at least in part on the anatomical characteristic(s). The controller may be further configured to compare the measured anatomical characteristic(s) with the corresponding anatomical characteristic(s) specified in the treatment plan; and vary one or more parameter values associated with the ultrasound transducer based on the comparison. In one implementation, the controller is further configured to vary the frequency associated with the ultrasound transducer among the two or more frequencies.

In some embodiments, the ultrasound transducer includes multiple transducer elements; the controller is further configured to group the transducer elements into multiple transducer groups, each group including at least some of the transducer elements and being different from the other groups. The transducer elements of one or more transducer groups may extend over a contiguous area. In addition, the controller may be further configured to cause the first one of the transducer groups to transmit the first series of ultrasound waves having the first frequency and the second one, different from the first one, of the transducer groups to transmit the second series of ultrasound waves having the second frequency. In one implementation, the transducer elements in each of the first one and the second one of the transducer groups form discrete areas. In additional, at least some of the discrete areas in the first and second transducer groups are interspersed.

In various embodiments, the transducer includes multiple transducer elements; the controller is further configured to cause the first and second series of ultrasound waves to be substantially simultaneously, sequentially or cyclically transmitted from the same or different transducer elements. Additionally, the controller may be further configured to cause the ultrasound transducer to transmit the first series and second series of ultrasound waves having an energy level above a predetermined level for target treatment. In one embodiment, the anatomical characteristic(s) includes a tissue acoustic parameter (e.g., tissue absorption and/or tissue impedance) and a change thereof resulting from the first series and second series of ultrasound waves.

In another aspect, the invention relates to a method of treating target tissue in a target volume having multiple target regions. In various embodiments, the method includes causing the first series of ultrasound waves having the first frequency to be transmitted to the first one of target regions; and causing the second series of ultrasound waves having the second frequency, different from the first frequency, to be transmitted to the second one of the target regions, different from the first one of the target regions, based on one or more anatomical characteristics differing between the first and second ones of the target regions. In one embodiment, the first frequency is higher than the second frequency and the anatomical characteristic is relative location; the location of the first target region corresponds to a shorter focal depth of the transducer than that of the second target region. In another embodiment, the first frequency is higher than the second frequency and the anatomical characteristic is vascularization; the first target region has higher vascularity than the second target region.

In various embodiments, the method further comprises measuring the anatomical characteristic(s) (e.g., the type, size, location, property, structure, thickness, density, and/or vascularization of tissue) associated with one or more target regions and/or a non-target region. In addition, the method may further include storing a treatment plan specifying the anatomical characteristic(s) and parameter values (e.g., the frequency, phase, amplitude and/or sonication duration) associated with the ultrasound transducer for transmitting the first series and second series of ultrasound waves based at least in part on the anatomical characteristic(s). The method may further include comparing the measured anatomical characteristic(s) with the corresponding anatomical characteristic(s) specified in the treatment plan; and varying the parameter values associated with the ultrasound transducer based on the comparison. In one implementation, the method further includes varying the frequency associated with the ultrasound transducer among the two or more frequencies.

In some embodiments, the first series and second series of ultrasound waves are transmitted from an ultrasound transducer including multiple transducer elements; the method further includes grouping the transducer elements into multiple transducer groups, each group including at least some of the transducer elements and being different from the other groups. The transducer elements of one or more transducer groups may extend over a contiguous area. In addition, the first series of ultrasound waves having the first frequency may be transmitted from the first one of the transducer groups and the second series of ultrasound waves having the second frequency may be transmitted from the second one, different from the first one, of the transducer groups. In one implementation, the transducer elements in each of the first one and the second one of the transducer groups form discrete areas. In addition, at least some of the discrete areas in the first and second transducer groups are interspersed.

In various embodiments, the first series and second series of ultrasound waves are transmitted from an ultrasound transducer including multiple transducer elements; the method further includes causing the first and second series of ultrasound waves to be substantially simultaneously, sequentially, or cyclically transmitted from the same or different transducer elements. Additionally, the method may further include causing the ultrasound transducer to transmit the first series and second series of ultrasound waves having an energy level above a predetermined level for target treatment. In one embodiment, the anatomical characteristic(s) includes a tissue acoustic parameter (e.g., tissue absorption and/or tissue impedance) and a change thereof resulting from the first series and second series of ultrasound waves.

Another aspect of the invention relates to a system for treating target tissue in a target region. In various embodiments, the system includes an ultrasound transducer for transmitting ultrasound waves having multiple frequencies; and a controller configured to determine two or more maximal angular steering ranges of an ultrasound beam at the target region; compute two or more frequencies of the ultrasound waves associated with the two or more maximal angular steering ranges; cause the ultrasound transducer to generate the first ultrasound beam having the first one of the computed frequencies; and cause the ultrasound transducer to generate the second ultrasound beam having the second one of the computed frequencies, different from the first one of the computed frequencies, so as to change the maximal angular steering range of the ultrasound beam.

In some embodiments, the controller is further configured to steer the first and/or second ultrasound beam in one orientation, two orientations, or three orientations. In addition, the system may further include an imaging system (e.g., an MRI apparatus) for acquiring an anatomical characteristic (e.g., the type, size, location, property, structure, thickness, density and/or vascularization of tissue) associated with the target region; the controller is further configured to determine the maximal angular steering ranges based at least in part on the acquired anatomical characteristic. In various embodiments, the ultrasound transducer includes multiple transducer elements; the controller is further configured to group the transducer elements into multiple transducer groups, each group having at least some of the transducer elements and being different from the other groups. In addition, the transducer elements of one or more transducer groups may extend over a contiguous area. In some embodiments, the controller is further configured to cause the first one of the transducer groups to transmit the first ultrasound beam and the second one, different from the first one, of the transducer groups to transmit the second ultrasound beam. In one implementation, the transducer elements in each of the first one and the second one of the transducer groups form discrete areas. In addition, at least some of the discrete areas in the first and second transducer groups are interspersed.

In various embodiments, the transducer includes multiple transducer elements; the controller is further configured to cause the first and second ultrasound beams to be substantially simultaneously, sequentially, or cyclically transmitted from the same or different transducer elements. In addition, the controller may be further configured to cause the ultrasound transducer to transmit the first and second ultrasound beams having an energy level above a predetermined level for target treatment.

In yet another aspect, the invention pertains to a method of treating target tissue in a target region. In various embodiments, the method includes determining two or more maximal angular steering ranges of an ultrasound beam at the target region; computing two or more frequencies of the ultrasound waves associated with the two or more maximal angular steering ranges; causing an ultrasound transducer to generate the first ultrasound beam having the first one of the computed frequencies; and causing the ultrasound transducer to generate the second ultrasound beam having the second one of the computed frequencies, different from the first one of the computed frequencies, so as to change the maximal angular steering range of the ultrasound beam.

In some embodiments, the method further includes steering the first and/or second ultrasound beam in one orientation, two orientations, or three orientations. In addition, the method may further include acquiring an anatomical characteristic (e.g., the type, size, location, property, structure, thickness, density and/or vascularization of tissue) associated with the target region; the maximal angular steering ranges are determined based at least in part on the acquired anatomical characteristic. In various embodiments, the ultrasound transducer includes multiple transducer elements; the method further includes grouping the transducer elements into multiple transducer groups, each group including at least some of the transducer elements and being different from the other groups. In addition, the transducer elements of one or more transducer groups may extend over a contiguous area. In some embodiments, the method further includes causing the first one of the transducer groups to transmit the first ultrasound beam and causing the second one, different from the first one, of the transducer groups to transmit the second ultrasound beam. In one implementation, the transducer elements in each of the first one and the second one of the transducer groups form discrete areas. In addition, at least some of the discrete areas in the first and second transducer groups are interspersed.

In various embodiments, the transducer includes multiple transducer elements; the method further includes causing the first and second ultrasound beams to be substantially simultaneously, sequentially, or cyclically transmitted from the same or different transducer elements. In addition, the method may further include causing the ultrasound transducer to transmit the first and second ultrasound beams having an energy level above a predetermined level for target treatment.

As used herein, the term “substantially” means ±10%, and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Moreover, the terms “focal depth” and “focal length” are used herein interchangeably. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIGS. 1A-1C schematically illustrate exemplary focused ultrasound systems in accordance with various embodiments;

FIG. 2A depicts an exemplary configuration of the transducer elements for directing ultrasound waves having different frequencies to different regions of a target volume in accordance with various embodiments;

FIGS. 2B and 2C are a flow charts illustrating exemplary approaches for applying ultrasound waves having different frequencies to different regions of a target volume in accordance with various embodiments;

FIG. 3A depicts adjustments of the transducer settings based on real-time thermal feedback in accordance with various embodiments;

FIG. 3B is a flow chart illustrating an exemplary approach for executing and modifying a treatment plan in accordance with various embodiments;

FIG. 4 is a flow chart illustrating an exemplary approach for optimizing one or more parameters of the sonications for treating one or more target regions in a target volume in accordance with various embodiments;

FIG. 5A illustrates the principle of electronic steering of a two-dimensional planar transducer array having multiple transducer elements in accordance with various embodiments;

FIG. 5B schematically illustrates lateral steering of an acoustic beam via adjustments of the transducer settings in accordance with various embodiments; and

FIG. 5C is a flow chart illustrating an exemplary approach for providing an acoustic beam having a desired steering angle and steering accuracy in accordance with various embodiments.

### DETAILED DESCRIPTION

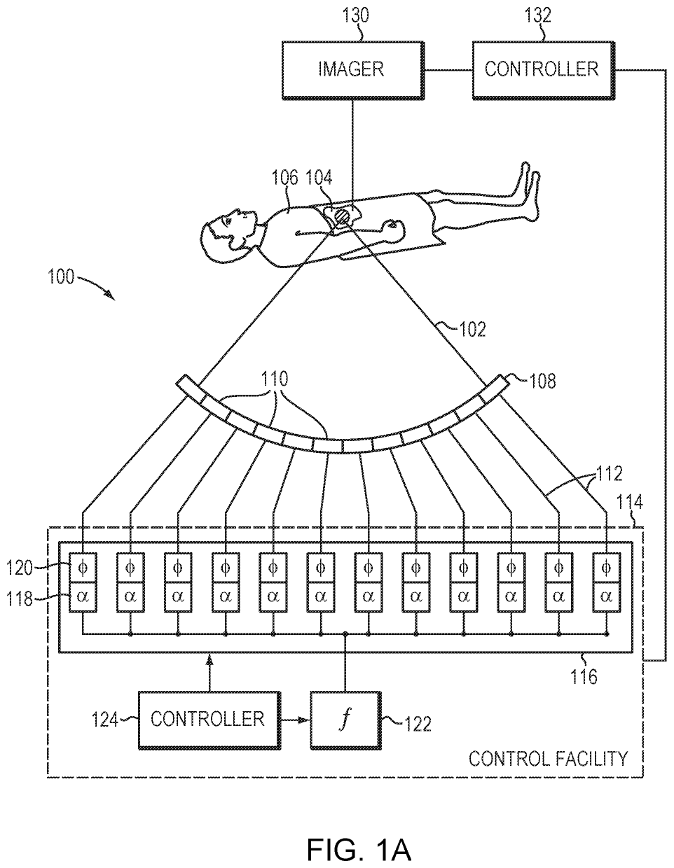

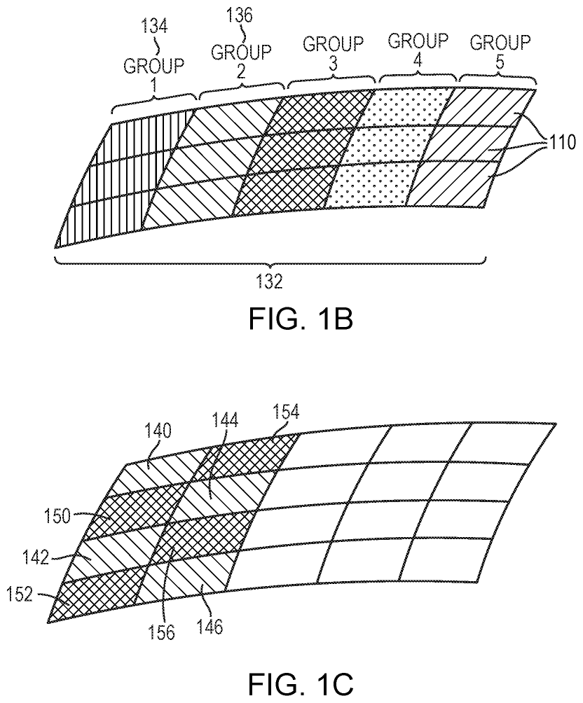

FIG. 1A is a simplified schematic representation of an exemplary focused ultrasound system 100 used to generate and deliver a focused acoustic energy beam 102 to a targeted volume 104 in a patient 106. The system 100 employs an ultrasound transducer 108 that is geometrically shaped and physically positioned relative to the patient 106 in order to focus the ultrasonic energy beam 102 at a three-dimensional focal zone located within the targeted volume 104. The system can shape the ultrasonic energy in various ways, producing, for example, a point focus, a line focus, a ring-shaped focus, or multiple foci simultaneously. The transducer 108 may be substantially rigid, semi-rigid, or substantially flexible, and can be made from a variety of materials, such as ceramics, plastics, polymers, metals, and alloys. The transducer 108 can be manufactured as a single unit, or, alternatively, may be assembled from a plurality of components (cells). While the illustrated transducer 108 has a “spherical cap” shape, a variety of other geometric shapes and configurations may be employed to deliver a focused acoustic beam, including other non-planar as well as planar (or linear) configurations. The dimensions of the transducer may vary, depending on the application, between millimeters and tens of centimeters.

In various embodiments, the transducer 108 delivers a high-power output with a desired transmission and reception frequency response profile. For example, the transducer 108 may generate ultrasound waves having multiple working frequencies; systems and methods for manufacturing and configuring the transducer to provide multiple frequencies and high-power output are described, for example, in U.S. Patent Publ. No. 2016/0114193, the entire disclosure of which is hereby incorporated by reference. In various embodiments, the transducer 108 includes a large number of transducer elements 110 arranged in a one-, two- or three-dimensional array or other regular manner, or in a random fashion. These elements 110 convert electronic drive signals into mechanical motion and, as a result, into acoustic waves. They may be made, for example, of piezoelectric ceramics or piezo-composite materials, and may be mounted in silicone rubber or another material suitable for damping the mechanical coupling between the elements 110. The transducer elements 110 are connected via electronic drive signal channels 112 to a control facility 114, which drives the individual transducer elements 110 so that they collectively produce a focused ultrasonic beam. More specifically, the control facility 114 may include a beamformer 116 that sets the frequencies and/or relative amplitudes and phases of the drive signals in channels 112. In conventional focused ultrasound systems containing n transducer elements, the beamformer 116 typically contains n amplifiers 118 and n phase control circuits 120, each pair driving one of the transducer elements 110. The beamformer 116 receives a radio frequency (RF) input signal, typically in the range from 0.1 MHz to 5 MHz, from a frequency generator 122. The input signal may be split into n channels for the n amplifiers and phase circuits 118, 120 of the beamformer 116. Thus, in typical systems, the radio frequency generator 122 and the beamformer 116 are configured to drive the individual elements 110 of the transducer 108 at the same frequency, but at different phases and different amplitudes, such that the transducer elements 110 collectively form a phased array. In various embodiments, the amplitudes and phase shifts imposed by the beamformer 116 are computed in a controller 124.

In certain embodiments, the system 100 further includes an imager 130, such as a magnetic resonance imaging (MRI) device, a computer tomography (CT) device, a positron emission tomography (PET) device, a single-photon emission computed tomography (SPECT) device, or an ultrasonography device, for acquiring images of the target and/or non-target tissue. The acquired images may be processed by a controller 132 associated with the imaging apparatus (or, in some embodiments, the transducer controller 124) to automatically identify therein the location of the target and/or non-target tissue using suitable image-processing techniques. In addition, the controller 132/124 may process the images to determine the anatomical characteristics (e.g., the type, property, structure, thickness, density, etc.) of the target/non-target tissue. The imager 130 provides a set of two-dimensional (2D) images suitable for reconstructing a three-dimensional (3D) image of the target and/or non-target tissue from which the anatomical characteristics thereof can be inferred; alternatively, image acquisition may be three-dimensional. In some embodiments, the controller 124/132 computationally divides the target volume 104 into multiple 3D regions based on their focal lengths (i.e., distances that the ultrasound beams propagate through the tissue and a spacing material located between the transducer 108 and the patient 106 prior to reaching the target regions); the transducer element 110 may then direct ultrasound waves having different frequencies to different regions of the target volume as further described below.

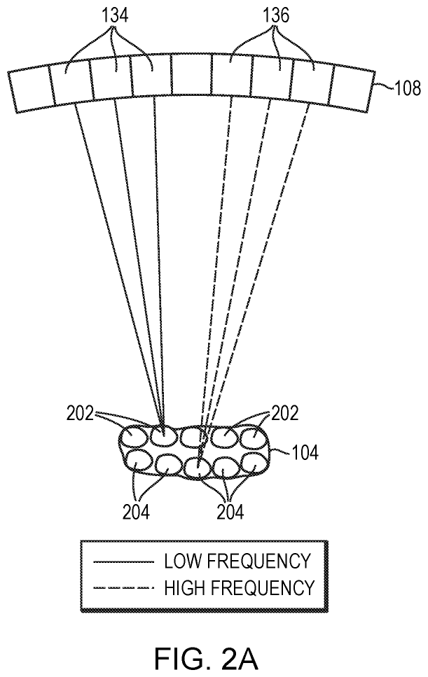

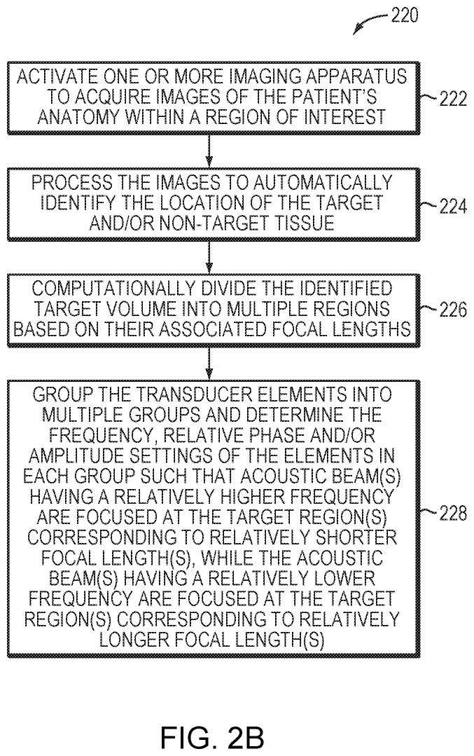

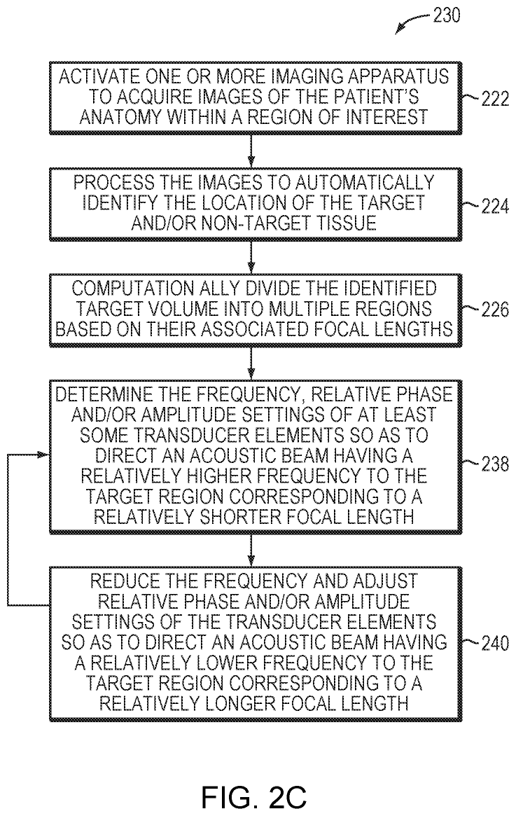

In some embodiments, the multi-frequency ultrasound waves are generated by multiple regions of the transducer elements. For example, referring to FIG. 1B, the control facility 114 may dynamically group the transducer elements 110 into multiple groups 132; each group 132 comprises or consists of a one- or two-dimensional array (i.e., a row or a matrix) of transducer elements 110. The transducer groups 132 may be separately controllable, i.e., they are each capable of emitting ultrasound waves at frequencies, amplitudes and/or phases that are independent of the frequencies, amplitudes and/or phases of the other groups 132. For example, referring to FIG. 2A, the control facility 114 may select one group 134 to collectively transmit a high-frequency ultrasound beam to one of the target regions 202 corresponding to a short focal depth and another group 136 to collectively transmit a low-frequency ultrasound beam to one of the target regions 204 corresponding to a long focal depth. Referring again to FIG. 1B, in one embodiment, the elements 110 in each transducer group may extend over a contiguous area, and the areas covered by different groups may or may not overlap. In another embodiment, with reference to FIG. 1C, the elements 110 in each group may form multiple discrete areas that are interspersed with each other. For example, the transducer group 134 that transmits the high-frequency ultrasound beam to the target regions 202 may form discrete areas 140-146 while the group 136 that transmits the low-frequency ultrasound beam to the target regions 204 may form discrete areas 150-156. It should be noted that the configurations of the transducer groups provided herein are for illustration only, and the present invention is not limited to such configurations. One of ordinary skill in the art will understand that many variations are possible and are thus within the scope of the present invention.

Referring again to FIG. 1A, the acoustic waves transmitted from the transducer elements 110 form the acoustic energy beam 102. Typically, the transducer elements 110 are driven so that the waves converge at a focal zone in the targeted volume 104. Within the focal zone, the acoustic power of the beam 102 is (at least partially) absorbed by the tissue, thereby generating heat and raising the temperature of the tissue to a point where the cells are denatured and/or ablated. The degree of ultrasound absorption for a propagation length in tissue is a function of frequency, given by:

Pₜ=P₀×(1−10^{-4fR})10^{-4f} Eq. (1),

where P₀ represents the initial acoustic power of ultrasound beams emitted from the transducer 108, f represents the frequency of the ultrasound (measured in MHz), a represents the absorption coefficient at the relevant frequency range (measured in cm⁻¹·MHz⁻¹) and may be acquired from known literature, R represents the focal length (which is measured in cm), and Pₜ represents the acoustic power at the target volume 104. Accordingly, as the focal depth, R, increases, the absorption of the acoustic power, Pₜ, in the focal zone decreases. In some embodiments, the reduced power absorption is compensated for by reducing the frequency, f, of the ultrasound waves as further described below.

The goal of a focused-ultrasound treatment is generally to maximize the acoustic power absorbed at the target 104 while minimizing the exposure of healthy tissue surrounding the target, as well as tissues along the beam path between transducer and target 104. To achieve this goal, with reference to FIG. 2A, the target volume 104 may be divided into multiple regions; the transducer may then substantially simultaneously, sequentially or cyclically direct ultrasound waves having different frequencies to different regions of the target. In one implementation, ultrasound waves having a high frequency (e.g., 3 MHz) are directed to the regions 202 corresponding to relatively shorter focal lengths, while ultrasound waves having a low frequency (e.g., 1.2 MHz) may be directed to the regions 204 corresponding to relatively longer focal lengths. As a result, at the high frequency, the acoustic power is substantially absorbed in the regions 202; and at the low frequency, the acoustic power is substantially absorbed in the regions 204, while limiting the power absorption in the regions 202. Accordingly, by varying the frequency of the ultrasound waves based on the focal length of the target region in the target volume 104, the acoustic power can be optimally absorbed in various regions of the target volume, while avoiding overheating a specific region (which can be a target or non-target region).

FIGS. 2B and 2C depict exemplary approaches 220, 230 for directing ultrasound waves having different frequencies to different regions of the target volume 104 in accordance herewith. In a first step 222, an imaging apparatus is activated to acquire images of the patient's anatomy within a region of interest. The images may be 3D images or a set of 2D image slices suitable for reconstructing 3D images of the anatomic region of interest. In a second step 224, the images are processed by a controller associated with the imaging apparatus to automatically identify therein the location of the target and/or non-target volumes using suitable image-processing techniques. In a third step 226, the controller may computationally divide the identified target volume into multiple regions based on their associated focal lengths. This step may involve determining the position and orientation of the target volume relative to the ultrasound transducer. In one embodiment, different imaging modalities are utilized. For example, the spatial characteristics of the multiple regions in the target volume may be acquired using MRI, whereas the orientations and locations of the transducer elements may be obtained using, e.g., a time-of-flight approach in the ultrasound system. As a consequence, it may be necessary to register coordinate systems in different imaging modalities prior to computing the focal length associated with each region in the target volume. Exemplary registration approaches are provided, for example, in U.S. Pat. No. 9,934,570, the entire disclosure of which is hereby incorporated by reference.

In a fourth step 228, the transducer control facility 114 may group the transducer elements 110 into multiple groups as described above, and subsequently, determine the frequency, relative phase and/or amplitude settings of the elements in each group such that acoustic beam(s) having a relatively higher frequency (e.g., 3 MHz) are focused at the target region(s) corresponding to relatively shorter focal length(s), while the acoustic beam(s) having a relatively lower frequency (e.g., 1.2 MHz) are focused at the target region(s) corresponding to relatively longer focal length(s). In addition, the control facility 114 may operate one or more groups of transducer elements to sequentially, cyclically or substantially simultaneously generate the acoustic beams having the two different frequencies. Alternatively, the transducer may be operated without grouping. For example, referring to FIG. 2C, the control facility 114 may activate at least some transducer elements 110 to direct the acoustic beam having a relatively higher frequency (e.g., 3 MHz) to the target region(s) corresponding to a relatively shorter focal length (in step 238); subsequently, the control facility 114 may reduce the sonication frequency and adjust the relative phases and/or amplitudes of the activated transducer elements so as to generate a new acoustic beam having the reduced frequency at the target region(s) corresponding to a relatively longer focal length (in step 240). Steps 238 and 240 may be iteratively performed until a desired treatment effect at the target region(s) is achieved.

In various embodiments, prior to treatment, a treatment plan is determined based on, for example, anatomical characteristics (e.g., the type, size, location, property, structure, thickness, density, vascularization, etc.) of the target tissue and/or non-target tissue. The treatment plan may include, for example, parameters (e.g., amplitudes, phases, frequencies and/or sonication durations) of the ultrasound waves for generating one or more foci at one or more regions in the target volume 104, one or more target temperatures corresponding to the region(s) in the target volume 104, and/or a maximal temperature of the non-target tissue. Approaches to computationally generating a treatment plan based on the anatomical characteristics of the target/non-target tissue are provided, for example, in U.S. Patent Publication No. 2015/0359603 and International Application Nos. PCT/M2018/000834 (filed on Jun. 29, 2018) and PCT/M2017/001689 (filed on Dec. 13, 2017), the entire disclosures of which are hereby incorporated herein by reference.

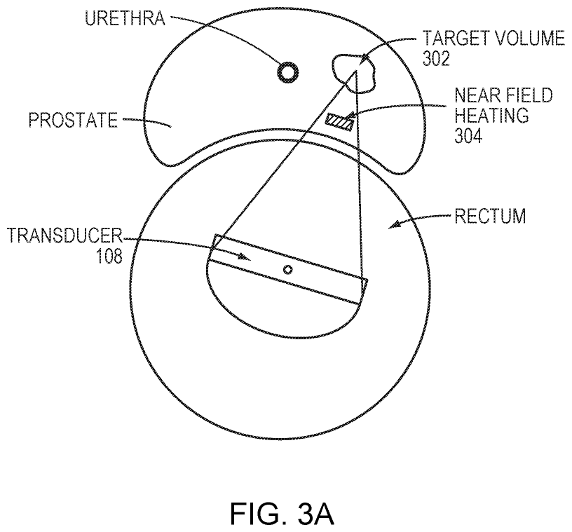

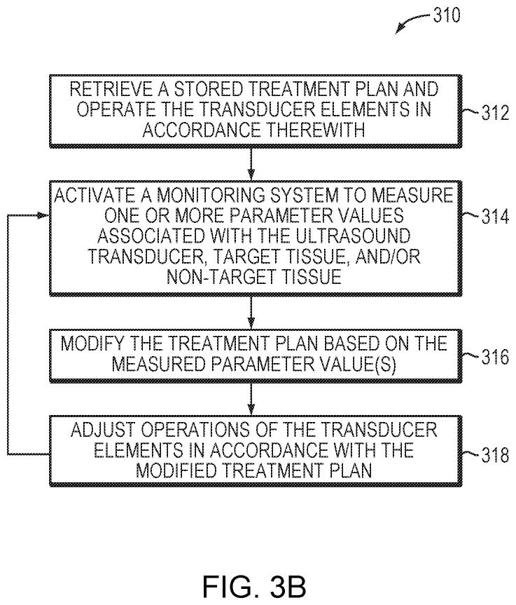

During treatment, the ultrasound system is activated and operated in accordance with the treatment plan. In addition, a monitoring system (e.g., an MRI apparatus 130) may in real-time measure the temperature at the target and/or non-target regions and provide the measured temperature to the control facility 114. The control facility 114 can then update the treatment plan based on the real-time feedback and cause the ultrasound system 100 to operate in accordance with the updated treatment plan, thereby optimizing treatment effects on the target region and avoiding damage to the non-target region. For example, with reference to FIG. 3A, the high-frequency waves may be first directed to initiate a treatment at the first target region 302. Upon detecting that the temperature in the second region 304, which is located in the near-field region and may be the target or non-target tissue, exceeds a predetermined threshold as specified in the treatment plan, the ultrasound system 100 may switch to a low-frequency mode for treatment so as to avoid overheating the second region 304.

FIG. 3B depicts an exemplary approach 310 for executing (and, in some embodiments, modifying) a treatment plan in accordance herewith in various embodiments. As shown, during the treatment procedure, the controller 124 may access memory storing the treatment plan and, based thereon, operate the transducer elements 110 (in a step 312). For example, the transducer elements 110 may be activated in accordance with the parameter values specified in the treatment plan to transmit high-frequency ultrasound waves/pulses focused at one or more target regions for treatment (e.g., thermal ablation). In a second step 314, the monitoring system may measure one or more parameter values associated with the ultrasound transducer, target tissue, and/or non-target tissue during treatment. For example, the monitoring system may include an imager for measuring a tissue characteristic (e.g., a temperature, a size, a shape or a location) of the target and/or non-target tissue in response to the sonication. In a third step 316, based on the measured parameter value(s), the control facility 114 may modify the treatment plan (e.g., the frequency of the applied ultrasound waves) to improve treatment efficiency and/or avoid damage to non-target tissue. Subsequently, operations of the transducer elements 110 may be adjusted in accordance with the modified treatment plan (step 318). Steps 314-318 may be iteratively performed throughout the entire treatment procedure.

Variations of the ultrasound frequency may also change the area of the focal zone at the target tissue, given by:

$$A = 2 {π ( 1.22 \times \frac{λ}{D} \times R )}^{2} , \qquad Eq . ( 2 )$$

where A represents the area of the focal zone for a circular transducer, A represents the wavelength of the ultrasound (λ=2π/j), D represents the diameter of the transducer elements, and R represents the focal length. In addition, the focal area, A, negatively correlates to the peak acoustic intensity, I, in the focal zone, satisfying:

I×A=Pₜ Eq. (3).

Therefore, at a given focal depth, increasing the ultrasound frequency may result in an increase of the peak acoustic intensity at the focal zone, which then increases the resulting temperature. Choice of the ultrasound frequency at a given focal depth thus reflects a trade-off between the absorption of acoustic power in the path zone, power absorption at the target, and the peak intensity at the focal zone. Accordingly, in various embodiments, the ultrasound frequency associated with each region in the target volume 104 is optimized based on the anatomical characteristics (e.g., the type, size, location, property, structure, thickness, density, vascularization, etc.) of the tissue therein. For example, if the target region includes highly vascular tissue that has a low absorption coefficient, a high ultrasound frequency may be applied thereto so as to increase the peak intensity at the focal zone without significantly reducing the acoustic power absorption therein. Approaches to determining an optimal frequency for treating the target tissue are provided, for example, in U.S. patent application Ser. No. 16/233,744 (filed on Dec. 27, 2018), the entire disclosure of which is hereby incorporated herein by reference. Additionally or alternatively, other parameters of the sonications (e.g., energy levels, durations of the sonications etc.) may be adjusted to optimize the treatment effect at the target region. For example, the high-power sonications may require ultrasound applications having short durations (e.g., a short sonication time). In some embodiments, the tissue acoustic parameter (such as tissue impedance and/or absorption) and a change thereof resulting from tissue interaction with the acoustic beam may be taken into account when determining the optimal frequency for treating each target region as well as the order of treating the target regions in the target volume. For example, because acoustic absorption of the coagulated tissue is relatively higher than that of the non-coagulated tissue, a higher sonication frequency may be necessary to effectively treat the target region that includes a relatively larger amount of non-coagulated tissue. In contrast, a lower sonication frequency may be sufficient to increase the temperature in the target region that includes a relatively larger amount of coagulated tissue for treatment. Likewise, when a relatively larger amount of coagulated tissue is located in the beam path zone, a lower sonication frequency may be applied to avoid excessive energy absorption by the non-target tissue in the beam path zone. Accordingly, by adjusting the frequency and/or other parameters of the ultrasound waves, the present invention accommodates tissue variability in the ultrasound procedure and thereby allows the acoustic power to be optimally and efficiently absorbed in various types of target regions.

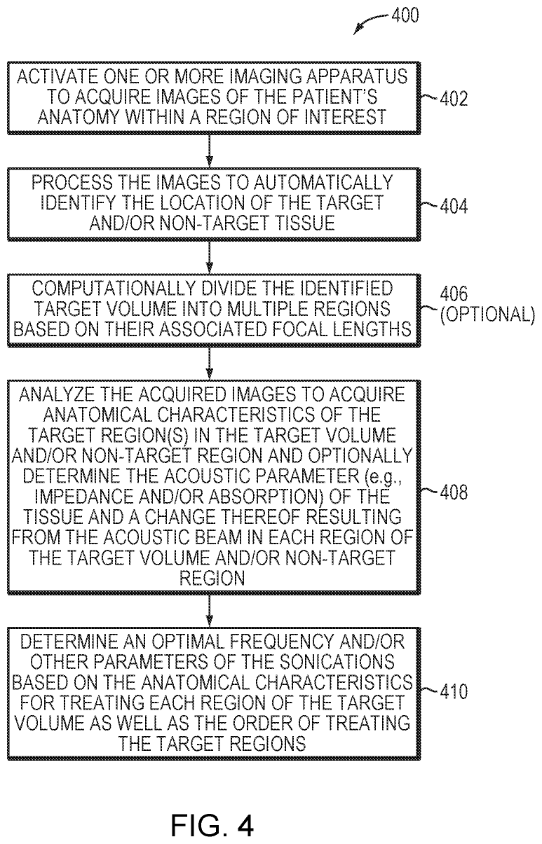

FIG. 4 depicts an exemplary approach 400 for optimizing one or more parameters (e.g., frequency) of the sonications for treating one or more target regions in the target volume in accordance herewith. In a first step 402, an imaging apparatus is activated to acquire images of the patient's anatomy within a region of interest. In a second step 404, the images are processed by a controller associated with the imaging apparatus to automatically identify therein the location of the target and/or non-target volumes using suitable image-processing techniques. In an optional step 406, the controller may computationally divide the identified target volume into multiple regions based on their associated focal lengths. Again, this step may involve different imaging modalities, and as a result, registration of the coordinate systems in different imaging modalities may be necessary (and may be achieved conventionally). In step 408, the acquired images may be analyzed to acquire the anatomical characteristics (e.g., the type, size, property, structure, thickness, density, vascularization, etc.) of the tissue in each region of the target volume and/or non-target region. Additionally, the control facility 114 may analyze the acquired images to determine the acoustic parameter (e.g., impedance and/or absorption) of the tissue and a change thereof resulting from the acoustic beam in each region of the target volume and/or non-target region. In step 410, based on the anatomical characteristics, the control facility 114 may determine the optimal frequency and/or other parameters of the sonications (e.g., energy levels, durations of the sonications, etc.) for treating each region of the target volume as well as the order of treating the target regions.

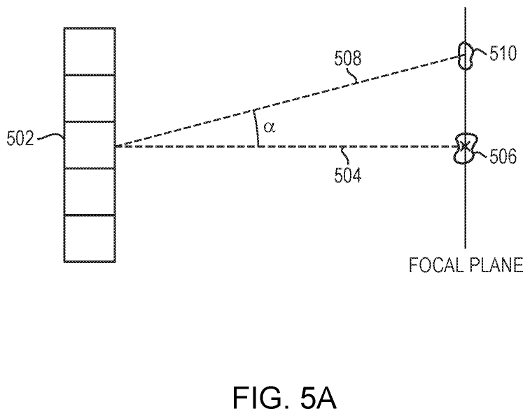

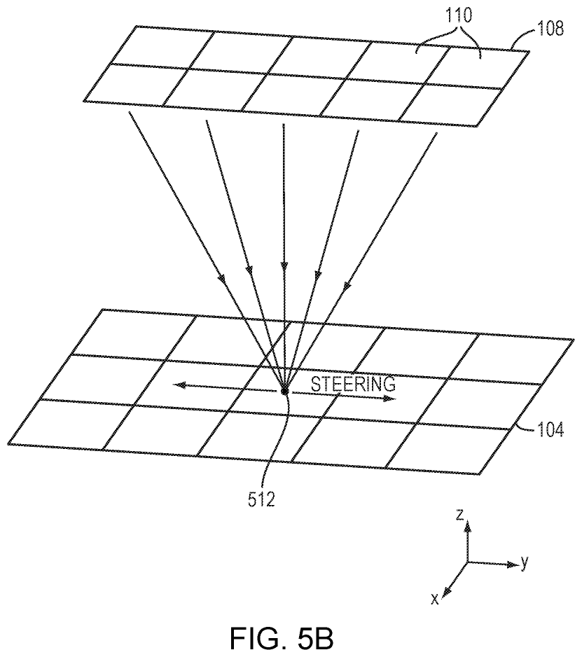

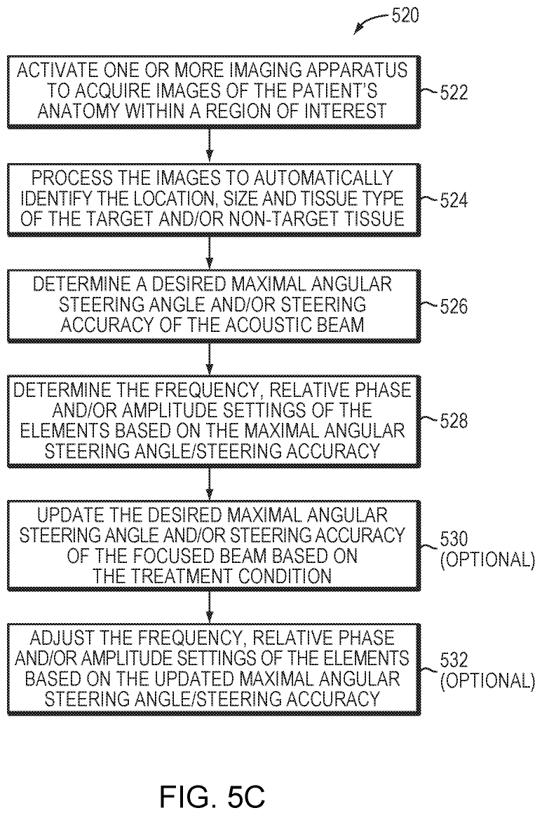

The location, shape, and intensity of the focal zone of the acoustic beam 102 is determined, at least in part, by the physical arrangement of the transducer elements 110, the physical positioning of the transducer 108 relative to the target volume 104, the structure and acoustic material properties of the tissues along the beam path between the transducer 108 and the target volume 104, and/or the frequencies, phase shifts and/or amplitudes of the drive signals. As noted above, “electronic steering” of the beam 102 is achieved by setting the drive signals so as to focus the acoustic energy at a desired location. FIG. 5A illustrates the principle of electronic steering of a two-dimensional planar transducer array that includes multiple transducer elements 502. In particular, the “steering angle” of any one transducer element of the array is the angle α between the first focal axis 504, extending generally orthogonally from the element, to an “unsteered” focal zone 506 at which the element 502 contributes a maximum possible power; and a second focal axis 508 extending from the transducer element 502 to a “steered-to” focal zone 510 located at the target volume. The “steering ability” of the transducer array is defined as a steering angle α at which energy delivered to the steered-to focal zone 510 falls to half of the maximum power delivered to the unsteered focal zone 506. Notably, the steering angle α of each transducer element of a phased array may be different, but as the distance from the elements to the focal zone increases, the respective steering angles for the array elements approach the same value. In practice, because the distance between the transducer array and the target volume is sufficiently longer than the distance between the transducer elements, the steering angles associated with the transducer elements in the array can be considered the same. Generally, the steering angle α of the beam 102 depends on the frequency of the waves. This is because the interference pattern of the acoustic beam at the target/non-target region is determined based on the shape and size of the transducer elements 110 as well as the wavelength of the ultrasound waves. Typically, the interference pattern includes a main lobe and side lobes having high directionality—the intensity of the lobes falls to zero at the steering angles: α=±1.22×λ/D degrees in the case of a circular transducer. Accordingly, the high-frequency ultrasound waves may have a more accurate but limited steering capability (e.g., α<±10°); whereas the low-frequency ultrasound waves may have a larger steering capability (e.g., α>) 30°.

In various embodiments, the need for a mechanical steering mechanism implemented in conventional ultrasound systems is obviated, or its required capabilities are reduced, using the transducer capable of generating multiple-frequency waves. For example, referring to FIG. 5B, the control facility 114 may drive the transducer elements 110 to generate an ultrasound beam 512 focused at the target volume 104 and to facilitate lateral steering of that beam in a direction perpendicular to beam propagation (e.g., along z axis). If a large steering angle (e.g., θ>±30°) is desired (e.g., when the target spans a large volume), the control facility 114 may drive the transducer elements 110 to generate a low-frequency ultrasound beam. If, however, more accurate steering is preferred (e.g., when the tissue surrounding the target volume is a heat-sensitive and/or important organ), the control facility 114 may drive the transducer elements 110 to generate an ultrasound beam having a high frequency. Typically, the beam may be electronically steered in one, two or three dimensions (e.g., along the x axis, z axis and/or y axis). In one embodiment, the beam is electronically steered in one dimension (e.g., along the x axis) only, and the mechanical steering mechanism is utilized to steer the beam in the other dimension (e.g., along the y axis). Regardless of whether the transducer 108 provides one-dimensional, two-dimensional or three-dimensional steering (via adjustment of the ultrasound frequency), the transducer 108 can generate an ultrasound beam to steer various regions of the target volume 104 with the desired steering capability and accuracy.

FIG. 5C depicts an exemplary approach 520 for providing an acoustic beam having a desired steering angle and steering accuracy in accordance with various embodiments. In a first step 522, an imaging apparatus is activated to acquire images of the patient's anatomy within a region of interest. In a second step 524, the images are processed by a controller associated with the imaging apparatus to automatically identify therein the anatomical characteristics (e.g., location, size and/or tissue type) of the target and/or non-target volumes using suitable image-processing techniques. In a third step 526, based on the anatomical characteristics of the target/non-target volume, the control facility 114 may determine a desired maximal angular steering angle and/or steering accuracy of the acoustic beam. For example, when the target spans a large volume, a larger steering angle may be preferred. In addition, if the tissue surrounding the target volume is a heat-sensitive or important organ, a higher steering accuracy may be desirable. In a fourth step 528, based on the determined maximal angular steering angle/steering accuracy, the control facility 114 may determine the frequency (and other ultrasound parameters such as relative phase and/or amplitude settings) of the elements 110 for generating a focal zone at the target volume. In addition, the control facility 114 may optionally update the desired maximal angular steering angle and/or steering accuracy of the focused beam during the ultrasound procedure based on a treatment condition (e.g., a change in the size of the target volume or a change in the distance between the focal zone in the target volume and the non-target tissue) (step 530). Subsequently, the control facility 114 may adjust the frequency (and other ultrasound parameters) of the elements 110 for generating a focus having the updated, desired maximal angular steering angle and/or steering accuracy (step 532).

In general, functionality for delivering a high-power acoustic output with two or more frequencies to a target volume and/or adjusting the steering angle of the acoustic beam may be structured in one or more modules implemented in hardware, software, or a combination of both, whether integrated within a controller of ultrasound system 100 and/or the monitoring system 130, or provided by a separate external controller or other computational entity or entities. Such functionality may include, for example, analyzing imaging data of the target and/or non-target volumes acquired using the imager 130, determining the location and/or anatomical characteristics (such as the tissue type, size, location, tissue structure, thickness, density, vascularization, etc.) of the target/non-target volume, computationally dividing the target volume into multiple regions based on their associated focal lengths, grouping the transducer elements into multiple groups, determining the frequency, relative phase and/or amplitude settings of the elements in each transducer group for creating acoustic beam(s) having a relatively higher frequency at the target region(s) corresponding to relatively shorter focal length(s) and creating acoustic beam(s) having a relatively lower frequency at the target region(s) corresponding to relatively longer focal length(s), retrieving a treatment plan stored in memory, causing a monitoring system to measure one or more parameter values associated with the ultrasound transducer, target tissue, and/or non-target tissue during treatment, modifying the treatment plan based on the measured parameter value(s), adjusting operations of the transducer elements in accordance with the modified treatment plan, determining an optimal frequency and/or other parameters of the sonications based on the anatomical characteristics of each region of the target volume, determining a desired maximal angular steering angle and/or steering accuracy of the acoustic beam based on the location, size and/or tissue type of the target and/or non-target tissue, determining the frequency, relative phase and/or amplitude settings of the elements based on the desired maximal angular steering angle/steering accuracy, updating the desired maximal angular steering angle and/or steering accuracy of the focused beam based on the treatment condition, and adjusting the frequency, relative phase and/or amplitude settings of the elements based on the updated angular steering angle/steering accuracy as described above.

Values of the ultrasound parameters (e.g., frequencies, relative phases and/or amplitudes) for focusing and/or steering the acoustic beam in various target regions of the target volume 104 are determined in a control module of the controller 124 which may be separate from the ultrasound control facility 114 or may be combined with the ultrasound control facility 114 into an integrated system control facility. In addition, the ultrasound control facility 114 and the monitoring-system controller 132 may be implemented in a single, integrated control facility or form two or more stand-alone devices in communication therebetween. Further, the ultrasound control module and/or control facility 114 may include one or more modules implemented in hardware, software, or a combination of both. For embodiments in which the functions are provided as one or more software programs, the programs may be written in any of a number of high level languages such as PYTHON, FORTRAN, PASCAL, JAVA, C, C++, C#, BASIC, various scripting languages, and/or HTML. Additionally, the software can be implemented in an assembly language directed to the microprocessor resident on a target computer; for example, the software may be implemented in Intel 80×86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

In addition, the term “controller,” “control facility” or “control module” used herein broadly includes all necessary hardware components and/or software modules utilized to perform any functionality as described above; the controller may include multiple hardware components and/or software modules and the functionality can be spread among different components and/or modules.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A system for treating target tissue in a target volume comprising a plurality of target regions, the system comprising:
an ultrasound transducer for transmitting ultrasound waves having two or more frequencies; and
a controller configured to:
(a) cause the ultrasound transducer to transmit a first series of ultrasound waves having a first frequency to a first one of target regions; and
(b) cause the ultrasound transducer to transmit a second series of ultrasound waves having a second frequency, different from the first frequency, to a second one of the target regions, different from the first one of the target regions, based on at least one different anatomical characteristic between the first and second ones of the target regions.

2. The system of claim 1, wherein the first frequency is higher than the second frequency and the at least one anatomical characteristic is relative location, a location of the first target region corresponding to a shorter focal depth of the transducer than that of the second target region.

3. The system of claim 1, wherein the first frequency is higher than the second frequency and the at least one anatomical characteristic is vascularization, the first target region having higher vascularity than the second target region.

4. The system of claim 1, further comprising a monitoring system for measuring the at least one anatomical characteristic associated with at least one of the target regions and/or a non-target region.

5. The system of claim 4, wherein the at least one anatomical characteristic comprises one or more of a type, a size, a location, a property, a structure, a thickness, a density, or vascularization of tissue.

6. The system of claim 4, further comprising memory for storing a treatment plan specifying the at least one anatomical characteristic and parameter values associated with the ultrasound transducer for transmitting the first series and second series of ultrasound waves based at least in part on the at least one anatomical characteristic.

7. The system of claim 6, wherein the controller is further configured to:
compare the at least one measured anatomical characteristic with the corresponding at least one anatomical characteristic specified in the treatment plan; and
vary at least one of the parameter values associated with the ultrasound transducer based on the comparison.

8. The system of claim 7, wherein the parameter value comprises at least one of the frequency, a phase, an amplitude or a sonication duration associated with the ultrasound transducer.

9. The system of claim 8, wherein the controller is further configured to vary the frequency associated with the ultrasound transducer among the two or more frequencies.

10. The system of claim 4, wherein the monitoring system comprises a magnetic resonance imaging device.

11. The system of claim 1, wherein the ultrasound transducer comprises a plurality of transducer elements, the controller being further configured to group the transducer elements into a plurality of transducer groups, each group comprising at least some of the transducer elements and being different from the other groups.

12. The system of claim 11, wherein the transducer elements of at least one of the transducer groups extend over a contiguous area.

13. The system of claim 11, wherein the controller is further configured to cause a first one of the transducer groups to transmit the first series of ultrasound waves having the first frequency and a second one, different from the first one, of the transducer groups to transmit the second series of ultrasound waves having the second frequency.

14. The system of claim 13, wherein the transducer elements in each of the first one and the second one of the transducer groups form discrete areas.

15. The system of claim 14, wherein at least some of the discrete areas in the first and second transducer groups are interspersed.

16. The system of claim 1, wherein the transducer comprises a plurality of transducer elements, the controller being further configured to cause the first and second series of ultrasound waves to be substantially simultaneously transmitted from different transducer elements.

17. The system of claim 1, wherein the transducer comprises a plurality of transducer elements, the controller being further configured to cause the first and second series of ultrasound waves to be sequentially transmitted from different transducer elements.

18. The system of claim 1, wherein the transducer comprises a plurality of transducer elements, the controller being further configured to cause the first and second series of ultrasound waves to be cyclically transmitted from different transducer elements.

19. The system of claim 1, wherein the transducer comprises a plurality of transducer elements, the controller being further configured to cause the first and second series of ultrasound waves to be substantially simultaneously transmitted from the same transducer elements.

20. The system of claim 1, wherein the transducer comprises a plurality of transducer elements, the controller being further configured to cause the first and second series of ultrasound waves to be sequentially transmitted from the same transducer elements.

21. The system of claim 1, wherein the controller is further configured to cause the ultrasound transducer to transmit the first series and second series of ultrasound waves having an energy level above a predetermined level for target treatment.

22. The system of claim 1, wherein the at least one anatomical characteristic comprises a tissue acoustic parameter and a change thereof resulting from the first series and second series of ultrasound waves.

23. The system of claim 22, wherein the tissue acoustic parameter comprises at least one of tissue absorption or tissue impedance.

24-75. (canceled)
